(19) United States
(12) Patent Application Publication
(10) Pub. No.: US 2025/0352433 A1
(43) Pub. Date: Nov. 20, 2025

(54) SINGLE-DOSE PUMPING AND DISPENSING OF OPIOID ADDICTION MEDICATION

(71) Applicant: OPIO CONNECT, INC., Kalispell, MT (US)

(72) Inventors: Amber Norbeck, Kalispell, MT (US); Michael Pokorny, Kalispell, MT (US); Thomas Hoffmann, Kalispell, MT (US)

(21) Appl. No.: 19/211,820

(22) Filed: May 19, 2025

Related U.S. Application Data
(60) Provisional application No. 63/649,222, filed on May 17, 2024.

(51) Int. Cl. A61J 7/00; A61J 1/03; G07F 11/44

(52) U.S. Cl. CPC A61J 7/0076; A61J 1/03; A61J 7/0046; G07F 11/44

(57) ABSTRACT

A device for automated pumping and dispensing of single doses of opioid addiction medication to a user. The device includes a pumping platform, an outlet, and a cup dispensing component configured to dispense a cup onto the pumping platform. The device further includes a pumping mechanism configured to rotate and pump the opioid addiction medication into the cup. The device further includes a pusher wheel operatively coupled to the pumping platform, configured to cyclically rotate between positions. In one position, the pusher wheel pushes the pumping mechanism away and blocks the outlet. In another position, the pusher wheel moves such that the pumping mechanism rotates over the pumping platform and blocks the outlet. In another position, the pusher wheel pushes the cup from the pumping platform to the outlet and moves such that the outlet is exposed.

### CROSS-REFERENCE TO RELATED APPLICATIONS

This application is a non-provisional and claims benefit of U.S. Provisional Application No. 63/649,222 filed May 17, 2024, the specification of which is incorporated herein in its entirety by reference.

### FIELD OF THE INVENTION

The present invention is directed to automated pumping and dispensing of single doses of opioid addiction medication.

### BACKGROUND OF THE INVENTION

In correctional facilities, a nurse is required to be on staff to pour out and deliver medication for opioid addiction to incarcerated patients. This is a major bottleneck and may result in some patients not receiving their medication on time or at all. This is an even bigger issue in more remote areas where it is more difficult to get nurses certified for opioid addiction treatment on staff. Thus, there exists a present need for a system that allows for the secure pumping and dispensing of single doses of opioid addiction medication to patients in correctional facilities.

### BRIEF SUMMARY OF THE INVENTION

It is an objective of the present invention to provide devices that allow for automated pumping and dispensing of single doses of opioid addiction medication, as specified in the independent claims. Embodiments of the invention are given in the dependent claims. Embodiments of the present invention can be freely combined with each other if they are not mutually exclusive.

The present invention features a device for automated pumping and dispensing of single doses of opioid addiction medication to a user. The device may comprise a pumping platform and an outlet. The device may further comprise a cup dispensing component configured to hold a plurality of cups and dispense a cup of the plurality of cups onto the pumping platform. The device may further comprise a pumping mechanism configured to rotate between a pumping position and a stored position. In the pumping position, the pumping mechanism may be disposed over the pumping platform to pump the opioid addiction medication into the cup. In the stored position, the pumping mechanism may be turned away from the pumping platform.

The device may further comprise a pusher wheel operatively coupled to the pumping platform, configured to cyclically rotate on the axle from a first position to a second position to a third position. In the first position, the pusher wheel may be further configured to push the pumping mechanism into the stored position and block the outlet. In the second position, the pusher wheel may be further configured to move such that the pumping mechanism rotates into the pumping position, and block the outlet. In the third position, the pusher wheel may be further configured to push the cup from the pumping platform to the outlet and move such that the outlet is exposed. The device may further comprise a door operatively coupled to the pusher wheel and the outlet, configured to close when the pusher wheel is in the first position and the second position, and open when the pusher wheel is in the third position.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a view of the device for automated pumping and dispensing of single doses of opioid addiction medication to a user of the present invention.

FIG. 2 shows a view of the pusher wheel of the device of the present invention.

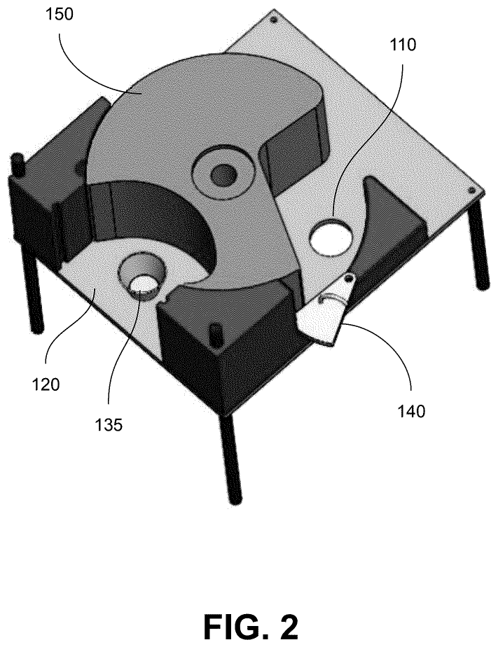

### DETAILED DESCRIPTION OF THE INVENTION

Following is a list of elements corresponding to a particular element referred to herein:
100 device
110 pumping platform
120 outlet
130 cup dispensing component
135 cup
140 pumping mechanism
150 pusher wheel
160 door

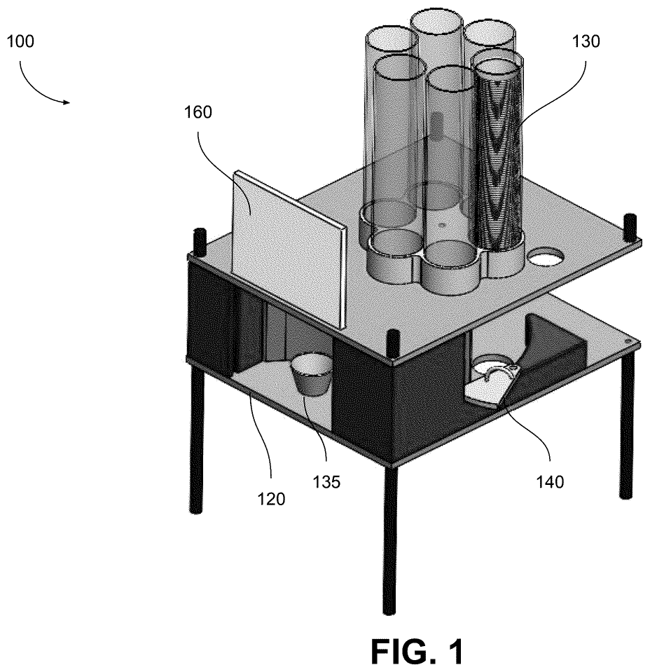

Referring now to FIG. 1, the present invention features a device (100) for automated pumping and dispensing of single doses of opioid addiction medication to a user. In some embodiments, the device (100) may comprise a pumping platform (110). The device (100) may further comprise an outlet (120). The device (100) may further comprise a cup dispensing component (130) disposed in line with the pumping platform (110), configured to hold a plurality of cups and dispense a cup (135) of the plurality of cups onto the pumping platform (110). The device (100) may further comprise a pumping mechanism (140) operatively coupled to the pumping platform (110), configured to rotate between a pumping position and a stored position. In the pumping position, the pumping mechanism (140) may be disposed over the pumping platform (110). In the stored position, the pumping mechanism (140) may be turned away from the pumping platform (110). In the pumping position, the pumping mechanism (140) may be further configured to pump the opioid addiction medication into the cup (135) on the pumping platform (110).

The device (100) may further comprise a pusher wheel (150) operatively coupled to the pumping platform (110), comprising a blocking portion, a hooking portion, and an axle disposed through a center of the pusher wheel (150). The pusher wheel (150) may be configured to cyclically rotate on the axle from a first position to a second position, to a third position. In the first position, the pusher wheel (150) may be further configured to push, by the blocking portion, the pumping mechanism (140) into the stored position and block, by the blocking portion, the outlet (120). In the second position, the pusher wheel (150) may be further configured to move the blocking portion such that the pumping mechanism (140) rotates into the pumping position, and block, by the blocking portion, the outlet (120). In the third position, the pusher wheel (150) may be further configured to push, by the hooking portion, the cup (135) from the pumping platform (110) to the outlet (120), and move the blocking portion such that the outlet (120) is exposed. The device (100) may further comprise a door (160) operatively coupled to the pusher wheel (150) and the outlet (120), configured to close when the pusher wheel (150) is in the first position and the second position, and open when the pusher wheel (150) is in the third position.

In some embodiments, the device (100) may further comprise a biometric authentication component. In some embodiments, the device (100) of the present invention may be configured to actuate upon receiving biometric confirmation from the biometric authentication component. In some embodiments, the biometric confirmation may comprise accepting biometric data from a user (e.g. fingerprints, retinal scan, facial scan), comparing the biometric data to a database mapping biometric data to patient identities, and actuating the device (100) if a patient identity matching the biometric data was found. In some embodiments, the database may further comprise dosage information (e.g. dosage type, dosage size). In some embodiments, the cup dispensing component may be configured to actuate upon receiving the biometric confirmation. In some embodiments, the pumping mechanism may be configured to actuate upon receiving the biometric confirmation. In some embodiments, the pumping mechanism may be further configured to dispense the medication into the cup according to the dosage information. In some embodiments, the door component may be configured to actuate upon receiving the biometric confirmation. In some embodiments, the device (100) may be configured to biometrically release the dose to the user after their identity has been verified.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A device (100) for automated pumping and dispensing of single doses of opioid addiction medication to a user comprising:
a. a pumping platform (110);
b. an outlet (120);
c. a cup dispensing component (130) disposed in line with the pumping platform (110), configured to hold a plurality of cups, and dispense a cup (135) of the plurality of cups onto the pumping platform (110);
d. a pumping mechanism (140) operatively coupled to the pumping platform (110), configured to rotate between a pumping position and a stored position;
wherein, in the pumping position, the pumping mechanism (140) is disposed over the pumping platform (110);
wherein, in the stored position, the pumping mechanism (140) is turned away from the pumping platform (110);
wherein, in the pumping position, the pumping mechanism (140) is further configured to pump the opioid addiction medication into the cup (135) on the pumping platform (110);
e. a pusher wheel (150) operatively coupled to the pumping platform (110), comprising a blocking portion, a hooking portion, and an axle disposed through a center of the pusher wheel (150);
wherein the pusher wheel (150) is configured to cyclically rotate on the axle from a first position, to a second position, to a third position;
wherein, in the first position, the pusher wheel (150) is further configured to push, by the blocking portion, the pumping mechanism (140) into the stored position and block, by the blocking portion, the outlet (120);
wherein, in the second position, the pusher wheel (150) is further configured to move the blocking portion such that the pumping mechanism (140) rotates into the pumping position and block, by the blocking portion, the outlet (120);
wherein, in the third position, the pusher wheel (150) is further configured to push, by the hooking portion, the cup (135) from the pumping platform (110) to the outlet (120), and move the blocking portion such that the outlet (120) is exposed; and
f. a door (160) operatively coupled to the pusher wheel (150) and the outlet (120), configured to close when the pusher wheel (150) is in the first position and the second position, and open when the pusher wheel (150) is in the third position.
